(19) United States
(12) Patent Application Publication
(10) Pub. No.: US 2021/0093578 A1
(43) Pub. Date: Apr. 1, 2021

(54) ENCAPSULATED NUTRITIONAL AND PHARMACEUTICAL COMPOSITIONS

(71) Applicant: Clover Corporation Limited, Victoria (AU)

(72) Inventors: Bo Wang, Parkinson (AU); Mek Chu Ting Chenbg, Calamvale (AU); Glenn Elliott, Casuarina (AU)

(21) Appl. No.: 16/608,740

(22) PCT Filed: Apr. 17, 2018
(86) PCT No.: PCT/AU2018/050384

§ 371 Date: Oct. 25, 2019

(30) Foreign Application Priority Data
Apr. 27, 2017 (AU) 2017901524

(51) Int. Cl. A61K 9/48; A61K 9/107; A61K 35/60; A61K 35/612; A61K 47/36; A23L 33/00; A23D 9/007; A23L 33/115

(52) U.S. Cl. CPC A61K 9/4816; A61K 9/107; A61K 35/60; A61K 35/612; A23L 33/115; A61K 9/4833; A23L 33/30; A23D 9/007; A61K 47/36

(57) ABSTRACT

Provided herein are encapsulated compositions comprising one or more long chain polyunsaturated fatty acids (LCPUFAs) and at least one hydrocolloid, wherein the composition has a surface free fat content of less than about 5%. Also provided are methods for stabilising emulsions comprising one or more LCPUFAs and for increasing the efficiency of encapsulation of compositions comprising one or more LCPUFAs, the methods comprising incorporating at least one hydrocolloid into the emulsions or compositions.

### TECHNICAL FIELD

The present invention broadly relates to stable encapsulated compositions of phospholipid-containing oils or lipid compositions suitable for both nutritional and pharmaceutical applications.

### BACKGROUND OF THE DISCLOSURE

It is well known that long-chain polyunsaturated fatty acids (LCPUFAs) are an important nutritional component of the human diet and that many people fail to consume an adequate amount of these essential fatty acids, in particular omega-3 fatty acids such as eicosapentaenoic acid (EPA) and docosahexaenoic acid (DHA). A large number of studies have found that EPA and DHA play an influential role in heart, brain and eye health. For example, recent studies suggest that EPA and DHA may have the ability to decrease heart rate and oxygen consumption during exercise, therefore contributing to enhanced physical and mental performance in athletes. Because of their essential nutritional role, compositions comprising LCPUFAs such as EPA and DHA and are important in terms of both nutritional supplementation, and as pharmaceutical agents.

One of the problems associated with delivering LCPUFA's as a nutritional or pharmaceutical product is their susceptibility to oxidation under various conditions, leading to undesirable oxidation breakdown products which may adversely affect the organoleptic properties or physiological properties of the formulation. Hence, LCPUFAs are often stabilized by encapsulation. Emulsifying starches such as octenylsuccinic anhydride-modified starch in combination with carbohydrates offer a useful approach for stabilization of LCPUFAs, and the present applicant has previously demonstrated that beneficial amounts of LCPUFAs may be stabilised using amounts of octenylsuccinic anhydride-modified starch that comply with the relevant standards relating to various nutritional formulations such as infant formula using sources of reducing sugars, with dextrose equivalent values of between about 0 and 80 (WO2012/106777, the disclosure of which is incorporated herein by reference).

Studies have shown that providing long-chain fatty acids bound with phospholipids, such as LCPUFAs rich in phospholipid from marine, egg and plant sources, sphingomyelin and milk fat globule membrane from breast milk and dairy sources exhibit superior bioavailability of the fatty acids, due to the better adsorption into certain cell membranes of the human body, such as brain grey matter and the retina. Accordingly, there is increasing interest in the delivery of these phospholipid-rich lipids especially LCPUFAs in phospholipid-bound form in oils rich in phospholipids such as krill oil, fish oil and lipid extracts from marine species such as herring.

However, the preparation and encapsulation of compositions of such phospholipid-rich oils containing LCPUFAs can suffer from poor emulsion stability and inadequate micro-encapsulation efficiency using existing encapsulation techniques, resulting in a high level of surface free fat when the emulsion is converted into a powder form. There is a need for the development of improved methods for formulating and encapsulating compositions comprising phospholipid-rich oils and improved stabilisation of compositions.

### SUMMARY OF THE DISCLOSURE

The present disclosure is predicated on the inventors' surprising discovery that the stability of emulsions comprising phospholipid-containing oils or lipid compositions can be improved, and the encapsulation efficiency of such compositions can be increased, by the addition of a hydrocolloid.

A first aspect of the present disclosure provides an encapsulated composition comprising one or more LCPUFAs and at least one hydrocolloid wherein the composition has a surface free fat content of less than about 5%.

The composition may be an oil or lipid composition comprising the one or more LCPUFAs.

In an exemplary embodiment the composition has a surface free fat content of less than about 2%.

The composition may be in the form of an emulsion, such as on oil-in-water emulsion. The composition may be in the form of a powder, such as a spray dried powder.

Typically the oil or lipid composition is a phospholipid-containing oil or lipid composition, optionally a phospholipid-rich oil or lipid composition. The phospholipid-containing or phospholipid-rich oil or lipid composition may be naturally occurring or naturally derived, or may be synthetic. Optionally the one or more LCPUFAs are bound to the phosphate group of the phospholipid compound in the oil or lipid composition. The oil may comprise, for example, krill oil, a fish oil such as tuna oil, or an oil or lipid extract from the roe of one or more fish species such as herring. The one or more LCPUFAs may comprise DHA and/or EPA.

The at least one hydrocolloid may be present in a concentration of between about 0.05% to about 1% w/w or between about 0.1% to about 0.5% with respect to the amount of water in the composition. The at least one hydrocolloid may comprise an edible gum, such as xanthan gum. The xanthan gum may be present in a concentration of between about 0.05% to about 1% w/w or between about 0.1% to about 0.5% with respect to the amount of water in the composition.

The one or more LCPUFAs or the oil or lipid composition comprising the one or more LCPUFAs may be encapsulated, optionally with an octenylsuccinic anhydride-modified starch and two or more sources of reducing sugars. One of said sources of reducing sugars may have a dextrose equivalent (DE) value (DE) of between 20 and 60, and a second of said sources of reducing sugars may have a DE value of between about 0 and 20.

A second aspect of the present disclosure provides a method for increasing the efficiency of encapsulation of a composition comprising one or more LCPUFAs, the method comprising incorporating at least one hydrocolloid into said composition.

The composition may be an oil or lipid composition comprising the one or more LCPUFAs.

The composition may be in the form of an emulsion, such as on oil-in-water emulsion. The composition may be in the form of a powder, such as a spray dried powder product of an oil-in-water emulsion.

The efficiency of encapsulation may be determined and/or quantified by the surface free fat content of the encapsulated composition, compared to the surface free fat content in the absence of the at least one hydrocolloid. The surface free fat content of the composition, in the presence of the at least one hydrocolloid may be less than about 5% or less than about 2%.

The at least one hydrocolloid may be added before, with or after the encapsulant. The encapsulant may comprise an octenylsuccinic anhydride-modified starch and two or more sources of reducing sugars. Typically the at least one hydrocolloid and the encapsulant form a homogenous aqueous slurry.

The at least one hydrocolloid may be present in a concentration of between about 0.05% to about 1% w/w or between about 0.1% to about 0.5% with respect to the amount of water in the composition. The at least one hydrocolloid may comprise an edible gum, such as xanthan gum. The xanthan gum may be present in a concentration of between about 0.05% to about 1% w/w or between about 0.1% to about 0.5% with respect to the amount of water in the composition.

Typically the oil or lipid composition is a phospholipid-containing oil or lipid composition, optionally a phospholipid-rich oil or lipid composition. The phospholipid-containing or phospholipid-rich oil or lipid composition may be naturally occurring or naturally derived, or may be synthetic. Optionally the one or more LCPUFAs are bound to the phosphate group of the phospholipid compound in the oil or lipid composition. The oil may comprise, for example, krill oil, a fish oil such as tuna oil, or an oil or lipid extract from the roe of one or more fish species such as herring. The oil may also comprise an oil or lipid extract from egg, plant sources, sphingomyelin or milk fat globule membrane from breast milk or dairy sources. The one or more LCPUFAs may comprise DHA and/or EPA.

In a third aspect of the present disclosure there is provided a method for stabilising an emulsion comprising one or more LCPUFAs, the method comprising incorporating at least one hydrocolloid into said emulsion.

The emulsion may comprise an oil or lipid composition comprising the one or more LCPUFAs. The surface free fat content of the emulsion in the presence of the at least one hydrocolloid may be less than about 5% or less than about 2%.

Typically the emulsion is an oil-in-water emulsion. Typically the one or more LCPUFAs or the oil comprising one or more LCPUFAs is encapsulated. The at least one hydrocolloid may be added before, with or after the encapsulant. The encapsulant may comprise an octenylsuccinic anhydride-modified starch and two or more sources of reducing sugars. Typically the at least one hydrocolloid and the encapsulant form a homogenous aqueous slurry.

The at least one hydrocolloid may be present in a concentration of between about 0.05% to about 1% w/w or between about 0.1% to about 0.5% with respect to the amount of water in the emulsion. The at least one hydrocolloid may comprise an edible gum, such as xanthan gum. The xanthan gum may be present in a concentration of between about 0.05% to about 1% w/w or between about 0.1% to about 0.5% with respect to the amount of water in the emulsion.

In a fourth aspect of the present disclosure there is provided an emulsion stabilised according to the method of the third aspect.

In a fifth aspect of the present disclosure there is provided a stable emulsion comprising one or more LCPUFAs, wherein said emulsion further comprising at least one hydrocolloid.

Typically the emulsion is an oil-in-water emulsion.

Typically the oil or lipid composition is a phospholipid-containing oil or lipid composition, optionally a phospholipid-rich oil or lipid composition. The phospholipid-containing or phospholipid-rich oil or lipid composition may be naturally occurring or naturally derived, or may be synthetic. Optionally the one or more LCPUFAs are bound to the phosphate group of the phospholipid compound in the oil or lipid composition. The oil may comprise, for example, krill oil, a fish oil such as tuna oil, or an oil or lipid extract from the roe of one or more fish species such as herring. The oil may also comprise an oil or lipid extract from egg, plant sources, sphingomyelin or milk fat globule membrane from breast milk or dairy sources.

The at least one hydrocolloid may be present in a concentration of between about 0.05% to about 1% w/w or between about 0.1% to about 0.5% with respect to the amount of water in the emulsion. The at least one hydrocolloid may comprise an edible gum, such as xanthan gum. The xanthan gum may be present in a concentration of between about 0.05% to about 1% w/w or between about 0.1% to about 0.5% with respect to the amount of water in the emulsion.

The one or more LCPUFAs or the oil or lipid composition comprising the one or more LCPUFAs may be encapsulated, optionally with an octenylsuccinic anhydride-modified starch and two or more sources of reducing sugars. One of said sources of reducing sugars may have a dextrose equivalent (DE) value (DE) of between 20 and 60, and a second of said sources of reducing sugars may have a DE value of between about 0 and 20.

A sixth aspect of the present disclosure provides a composition comprising one or more LCPUFAs and at least one hydrocolloid.

The composition may be in the form of an emulsion, such as on oil-in-water emulsion. The composition may be in the form of a powder, such as a spray dried powder.

Typically the oil or lipid composition is a phospholipid-containing oil or lipid composition, optionally a phospholipid-rich oil or lipid composition. The phospholipid-containing or phospholipid-rich oil or lipid composition may be naturally occurring or naturally derived, or may be synthetic. Optionally the one or more LCPUFAs are bound to the phosphate group of the phospholipid compound in the oil or lipid composition. The oil may comprise, for example, krill oil, a fish oil such as tuna oil, or an oil or lipid extract from the roe of one or more fish species such as herring. The oil may also comprise an oil or lipid extract from egg, plant sources, sphingomyelin or milk fat globule membrane from breast milk or dairy sources.

The at least one hydrocolloid may be present in a concentration of between about 0.05% to about 1% w/w or between about 0.1% to about 0.5% with respect to the amount of water in the composition. The at least one hydrocolloid may comprise an edible gum, such as xanthan gum. The xanthan gum may be present in a concentration of between about 0.05% to about 1% w/w or between about 0.1% to about 0.5% with respect to the amount of water in the composition.

The one or more LCPUFAs or the oil or lipid composition comprising the one or more LCPUFAs may be encapsulated, optionally with an octenylsuccinic anhydride-modified starch and two or more sources of reducing sugars. One of said sources of reducing sugars may have a dextrose equivalent (DE) value (DE) of between 20 and 60, and a second of said sources of reducing sugars may have a DE value of between about 0 and 20.

### BRIEF DESCRIPTION OF THE FIGURES

Exemplary embodiments of the present disclosure are described herein, by way of non-limiting example only, with reference to the following drawings.

FIG. 1. Exemplary process flow of encapsulation of phospholipid-rich krill oil with protein-based Maillard reaction products, and with an octenylsuccinyl anhydride-modified starch-based hypoallergenic matrix with or without xanthan gum as described in Example 1.

### DETAILED DESCRIPTION

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” is understood to refer to a range of numbers that a person of skill in the art would consider equivalent to the recited value in the context of achieving the same function or result.

In the context of this specification, the terms “a” and “an” refer to one or to more than one (i.e. to at least one) of the grammatical object of the article. By way of example, “an element” means one element or more than one element.

As used herein, the term stable means, in relation to an emulsion that the emulsion does not exhibit phase separation for at least 48 hours after preparation of the emulsion. The emulsion is therefore said to exhibit stability.

In the context of this specification, the term “substantially free of protein” means that the amount of protein present in the composition is less than about 0.1%, or less than about 0.01%.

In the context of this specification, the term “hypoallergenic” is understood to mean that the composition to which it refers has a decreased likelihood of provoking an allergic reaction in a subject, and/or that the composition is free, or substantially free, of allergens.

Particular embodiments of the present disclosure provide emulsions and compositions comprising one or more long-chain polyunsaturated fatty acids (LCPUFAs) or an oil or lipid composition comprising one or more LCPUFAs, wherein said emulsion further comprising at least one hydrocolloid.

Compositions of the present disclosure may be in the form of a powder, and may be obtained by spray drying. In one embodiment, the composition is a free-flowing powder. The powder may have a mean particle size between about 10 μm and 1000 μm, or between about 50 μm and 800 μm, or between about 100 μm and 300 μm. In alternative embodiments the composition may be in the form of granules. Alternatively, the composition may be in the form of an emulsion, typically an oil-in-water emulsion.

Hydrocolloids are a heterogeneous group of long chain hydrophilic polymers, typically comprising a large number of hydroxyl groups, capable of forming viscous dispersions or gels in water. Any suitable hydrocolloid may be employed in accordance with the present disclosure. Particularly applicable are hydrocolloids used in the food and pharmaceutical industries such as starch, modified starch, xanthan gum, guar gum locust bean gum, gum Arabic, acacia gum, gum karaya, gum tragacanth, cellulose, carboxymethyl cellulose (CMC), pectin, agar, alginate, gelatin, gellan, arabinoxylan, β-glucan, carrageenan and curdlan. The hydrocolloid may be of animal, plant or microbial origin, or may be synthetically produced. In an exemplary embodiment, the hydrocolloid is xanthan gum.

The at least one hydrocolloid may be introduced into the emulsion or composition at any stage in the preparation of the emulsion or composition such that a homogenous aqueous dispersion or slurry is formed. In the case of encapsulated compositions, the at least one hydrocolloid may be introduced prior to the encapsulant, such as in the aqueous phase, at the same time as the encapsulant or after the encapsulant. Those skilled in the art will be able to optimise the amount of the at least one hydrocolloid to be introduced without undue burden or experimentation. The amount of the at least one hydrocolloid should be sufficient so as to produce a composition with the desired viscosity according to the application. In the case of oil-in-water emulsions, the viscosity should be sufficient to enable the emulsion to maintain the morphological structure of the oil-in-water droplets. If the hydrocolloid content is too low an unprotected encapsulation matrix may result, whereas if the hydrocolloid content is too high, the viscosity will be too great, hindering spray drying. Determining the appropriate hydrocolloid content and the appropriate viscosity is well within the capabilities of the skilled person.

In exemplary embodiments in which the hydrocolloid is xanthan gum, the xanthan gum may be present at between about 0.05% w/w to about 1% w/w, or between about 0.1% w/w and about 0.5% w/w with respect to the amount of water in the composition or emulsion. For example, the xanthan gum may be present at about 0.05%, 0.075%, 0.1%, 0.125%, 0.15%, 0.175%, 0.2%, 0.225%, 0.25%, 0.275%, 0.3%, 0.325%, 0.35%, 0.375%, 0.4%, 0.425%, 0.45%, 0.475%, 0.5%, 0.55%, 0.6%, 0.65%, 0.7%, 0.75%, 0.8%, 0.85%, 0.9%, 0.95% or 1% w/w with respect to the amount of water present.

Compositions and emulsions of the present disclosure comprise one or more LCPUFAs or an oil or a lipid composition comprising the one or more LCPUFAs. In particular embodiments the oil or lipid composition is a phospholipid-containing oil or lipid composition, more particularly a phospholipid-rich oil or lipid composition. Optionally at least a proportion of the one or more LCPUFAs are bound to the phosphate group of the phospholipid compound in the oil or lipid composition. A phospholipid-rich oil or lipid composition is one that may comprise at least about 5%, at least about 10%, at least about 15%, at least about 20%, at least about 25%, at least about 30%, at least about 35%, at least about 40%, at least about 45%, at least about 50%, or at least about 55% phospholipids.

The phospholipid-containing or phospholipid-rich oil or lipid composition, or oil or lipid composition modified so as to be phospholipid-containing or phospholipid-rich, may be present in a purified form and/or in the form of an extract from a suitable source. The source may be genetically modified or non-genetically modified. The oil or lipid composition may be naturally occurring or naturally derived, or may be synthetic. In the contest of the present disclosure “naturally occurring” and “naturally derived” includes oils and lipid compositions that may be extracted from a natural source such as the organisms listed herein, or that may be derived from or modified from an oil or one or more lipids found in such natural sources.

Exemplary oils that are, or can be modified to be, phospholipid-rich include oils from marine organisms such as, for example, crustaceans such as krill, molluscs such as oysters, and fish such as tuna, salmon, trout, sardines, mackerel, sea bass, menhaden, herring, pilchards, kipper, eel or whitebait. The oil may be from the roe of one or marine organisms such as those listed herein. In exemplary embodiments, the oil is or comprises hill oil or tuna oil or a lipid extract from fish roe.

Other exemplary oils that are, or may be modified to be phospholipid-rich, include plant sources and microbial sources. Plant sources include, but are not limited to, flaxseed, walnuts, sunflower seeds, canola, safflower, soy, wheat germ, corn and leafy green plants such as kale, spinach and parsley. Microbial sources include algae and fungi.

The oil or lipid composition may be present in an amount between about 0.1% and 80% of the total weight of the composition, or in an amount between about 1% and 80%, or in an amount between about 1% and 75%, or in an amount between about 5% and 80%, or in an amount between about 5% and 75%, or in an amount between about 5% and 70% of the total weight of the composition. In exemplary embodiments, where the oil is phospholipid-rich hill oil, the oil may be present in an amount of about 1%, 3%, 5%, 7%, 9%, 11%, 13%, 15%, 17%, 19%, 21%, 23%, 25%, 27%, 29%, 31%, 33%, 35%, 37%, 39%, 41%, 43%, 45%, 47%, 49%, 51%, 53%, 55%, 57%, 59%, 61%, 63%, 65%, 67%, 69%, 71%, 73% or 75% of the total weight of the composition .

The LCPUFAs typically comprise one or more omega-3 fatty acids and/or one or more omega-6 fatty acids, or mixtures thereof. The fatty acids may include DHA, AA, EPA, DPA and/or stearidonic acid (SDA), or mixtures thereof. In one embodiment, the fatty acids comprise DHA and AA. Where compositions and emulsions of the disclosure comprise DHA and AA, the DHA and AA may be present in a ratio between about 1:10 and 10:1, or in a ratio between about 1:5 and 5:1, or in a ratio between about 2:1 and 1:2, or in a ratio between about 1:1 and 1:5, or in a ratio between about 1:1 and 1:4, or in a ratio between about 1:1 and 1:3, or in a ratio between about 1:1 and 1:2, or in a ratio of about 1:1.

The present disclosure provides methods and compositions in which at least one hydrocolloid is used to increase encapsulation efficiency (for example, reducing or minimising surface free fat content) in an emulsion or dried powder derived from an emulsion and stabilising the emulsion. The surface free fat content may be reduced, in the presence of the hydrocolloid, to leas than about 10%, less than about 9%, less than about 8%, less than about 7%, less than about 6%, less than about 5%, less than about 4%, less than about 3%, less than about 2% or less than about 1%. In particular embodiments, this reduction in surface free fat content is seen in a powder derived or produced from an emulsion.

A variety of suitable encapsulation means or systems may be employed in accordance with the present disclosure. In one exemplary embodiment encapsulation comprises using octenylsuccinic anhydride-modified starch and one or more, or two or more, sources of reducing sugars, with dextrose equivalent values of between about 0 and 80 as has been described previously in WO2012/106777, the disclosure of which is incorporated herein by reference. Briefly, the starch may comprise primary and/or secondary modifications and may be an ester or half ester. Suitable octenylsuccinic anhydride-modified starches include, for example, those based on waxy maize and sold under the trade names PURITY GUM®, CAPSUL® IMF and HI CAP® IMF by National Starch and Chemical Pty Ltd, Seven Hills, NSW, Australia. The octenylsuccinic anhydride-modified starch may be present in an amount of less than about 18%, 17%, 16%, 15%, 14%, 13%, 12%, 11%, 10%, 9%, 8%, 7%, 6.5%, 6%, 5.5%, 5%, 4.5%, 4%, 3.5%, 3%, 2.5%, 2% or less than 1%, of the total weight of the composition. The octenylsuccinic anhydride-modified starch may be present in an amount between about 0.005% and 18%, or in an amount between about 1% and 18%, or in an amount between about 2% and 18%, or in an amount between about 3% and 18%, or in an amount between about 4% and 18%, or in an amount between about 5% and 18%, or in an amount between about 0.005% and 15%, or in an amount between about 0.5% and 10%, or in an amount between about 1% and 10%, or in an amount between about 1% and 9%, or in an amount between about 1% and 8%, or in an amount between about 1% and 7%, or in an amount between about 1% and 6%, or in an amount between about 1% and 5%, or in an amount between about 0.1% and 10%, or in an amount between about 0.1% and 8%, or in an amount between about 0.1% and 6%, of the total weight of the composition. Additional emulsifying starches may also be included as desired.

The at least one source of reducing sugars have a dextrose equivalent value between about 0 and 80. The at least one source of reducing sugars may have a dextrose equivalent value between about 0 and 80, 0 and 70, 0 and 60, 0 and 50, 0 and 40, 0 and 30, 0 and 20, 0 and 10, 1 and 20, 1 and 15, 1 and 10, 5 and 20 or 5 and 15. In a particular embodiment at least two sources of reducing sugars are employed, wherein a first source of reducing sugars has a dextrose equivalent value between 0 and 100, or between 0 and 80, or between 0 and 60, or between 10 and 60, or between 20 and 100, or between 20 and 80, or between 20 and 60, or between 20 and 50, or between 20 and 40, or between 25 and 40, or between 25 and 35, and the second source of reducing sugars has a dextrose equivalent value between 0 and 25, or between 0 and 20, or between 0 and 15, or between 5 and 15. In these embodiments, the weight ratio of the first source of reducing sugars to the second source of reducing sugars may be between about 1:10 and 10:1, or between about 1:6 and 6:1, or between about 1:5 and 5:1, or between about 1:1 and 1:10, or between about 1:1 and 1:8, or between about 1:1 and 1:6, or between about 1:1 and 1:5, or between about 1:1 and 1:4, or between about 1:2 and 1:10, or between about 1:2 and 1:8, or between about 1:2 and 1:6, or between about 1:2 and 1:5, or between about 1:3 and 1:10, or between about 1:3 and 1:8, or between about 1:3 and 1:6, or between about 1:4 and 1:10, or between about 1:4 and 1:8, or between about 1:4 and 1:6, or about 1:4.

In an embodiment, a first source of reducing sugars has a dextrose equivalent value between 20 and 60, and a second source of reducing sugars has a dextrose equivalent value between 0 and 20, wherein the first source of reducing sugars and the second source of reducing sugars are present in a ratio between about 1:1 and 1:10 by weight.

In another embodiment, a first source of reducing sugars has a dextrose equivalent value between 20 and 50, and a second source of reducing sugars has a dextrose equivalent value between 0 and 15, wherein the first source of reducing sugars and the second source of reducing sugars are present in a ratio between about 1:1 and 1:10 by weight.

In a further embodiment, a first source of reducing sugars has a dextrose equivalent value between 25 and 40, and a second source of reducing sugars has a dextrose equivalent value between 0 and 15, wherein the first source of reducing sugars and the second source of reducing sugars are present in a ratio between about 1:1 and 1:6 by weight.

In another embodiment, a first source of reducing sugars has a dextrose equivalent value between 20 and 40, and a second source of reducing sugars has a dextrose equivalent value between 5 and 15, wherein the first source of reducing sugars and the second source of reducing sugars are present in a ratio between about 1:1 and 1:6 by weight.

In still a further embodiment, a first source of reducing sugars has a dextrose equivalent value between 25 and 35, and a second source of reducing sugars has a dextrose equivalent value between 5 and 15, wherein the first source of reducing sugars and the second source of reducing sugars are present in a ratio between about 1:2 and 1:6 by weight.

In another embodiment, a first source of reducing sugars has a dextrose equivalent value of about 30, and a second source of reducing sugars has a dextrose equivalent value of about 10, wherein the first source of reducing sugars and the second source of reducing sugars are present in a ratio between about 1:2 and 1:6, or about 1:4.

Sources of reducing sugars are well known to those skilled in the art and include monosaccharides and disaccharides, for example glucose, fructose, maltose, galactose, glyceraldehyde and lactose. Suitable sources of reducing sugars also include oligosaccharides, for example glucose polymers, such as dextrin and maltodextrin and glucose syrup solids. The reducing sugars may also be derived from glucose syrup which typically contains not less than 20% by weight of reducing sugars.

The source(s) of reducing sugars may be present in an amount between about 10% and 80% of the total weight of the composition, or in an amount between about 10% and 75%, or in an amount between about 10% and 70%, or in an amount between about 15% and 70%, or in an amount between about 20% and 70%, or in an amount between about 25% and 65%, or in an amount between about 25% and 60%, or in an amount between about 30% and 65%, or in an amount between about 35% and 65%, or in an amount between about 40% and 65%, or in an amount between about 45% and 65%, or in an amount between about 50% and 65%, or in an amount between about 50% and 60%, of the total weight of the composition.

The source(s) of reducing sugars and the octenylsuccinic anhydride-modified starch may be present in the compositions in a ratio between about 3:1 and 15:1, or between about 4:1 and 14:1, or between about 4:1 and 13:1, or between about 5:1 and 15:1, or between about 7:1 and 15:1, or between about 8:1 and 14:1, or between about 8:1 and 12:1, or between about 8:1 and 11:1, or between about 10:1 and 11:1, by weight.

The compositions may be prepared by forming an aqueous mixture comprising the LCPUFAs, or oil or lipid composition containing the LCPUFAs, the source(s) of reducing sugars and octenylsuccinic anhydride-modified starch, and drying the mixture, for example, by spray drying. In an exemplary, the compositions may be prepared by solubilising the source(s) of reducing sugars and the octenylsuccinic anhydride-modified starch in an aqueous phase using a high shear mixer. The mixture may then be heated to a temperature of about 65° C. to 70° C. after which time one or more antioxidants may be added if desired. The LCPUFAs or oil may be dosed in-line to the aqueous mixture which is passed through a high shear mixer to form a coarse emulsion. The coarse emulsion may then be passed through homogenisation at 240/40 bar. If it is desired to prepare a powdered product the coarse emulsion may be pressurised and spray-dried at an inlet temperature of about 180° C. and an outlet temperature of 80° C. The at least one hydrocolloid may be introduced with the modified starch and the sugars, or may be added later during agitation, provided that a homogeneous aqueous slurry is produced.

Alternative means and systems for encapsulation are also contemplated. For example, any protein useful in encapsulating oils can be used. A carbohydrate with a reducing sugar functional group may be reacted with the protein. The protein is typically soluble and needs to be stable in the heating range of the Maillard reaction and includes casein, soy and whey proteins, gelatine, egg albumin and hydrolysed proteins with increased free amino acid groups including soy protein hydrolysate. In an embodiment the protein may be selected from sodium caseinate, whey protein isolate (WPI), soy protein isolate (SPI), skim milk powder (SMP), hydrolysed casein (HCP) and hydrolysed whey protein (HWP) and the carbohydrate, either alone or in combination, may be selected from dextrose (including dextrose monohydrate), glucose, lactose, sucrose, oligosaccharide and dried glucose syrup. In a further embodiment a polysaccharide, high-methoxy pectin or carrageenan, may be added to protein-carbohydrate mixtures in some formulations. Care needs to be taken in reacting the protein and carbohydrate to ensure that the conditions do not result in extensive gelling or coagulation of the protein, as this will render the protein incapable of forming a good film. In an embodiment the formation of the Maillard reaction product occurs with substantially no coagulation product being formed. In another embodiment the formation of the Maillard reaction product occurs with the formation of the coagulation product not exceeding greater than 5% of the product. In this regard it will be appreciated that the determination of the formation of Maillard reaction product can be achieved and therefore regulated by quantitative colourimetric analysis using an IR/UV spectrometer.

In an embodiment the protein may be a milk protein such as casein or whey protein isolate. Casein or a salt thereof such as sodium caseinate is a desirable protein in many applications because of its low cost and its greater resistance to gelling during the heat treatment to form the Maillard reaction products. The carbohydrate is a sugar with a reducing group optionally selected from the group consisting of monosaccharides (e.g. dextrose, (including dextrose monohydrate) glucose, fructose), disaccharides (e.g. maltose, lactose), trisaccharides, oligosaccharides and glucose syrups, and mixtures thereof. Any suitable reducing sugar source may be used, including honey.

The amount of Maillard reaction product in the protein-carbohydrate mixture is an amount sufficient to provide antioxidant activity for the period of the product's shelf life is needed. Preferably the minimum reaction required between the protein and carbohydrate prior to encapsulation consumes at least 5% of the sugar present, for instance, at least 6%, for instance at least 7%, for instance at least 8%, for instance at least 9%, or for instance at least 10%, of the sugar present. The extent of Maillard reaction product formed can be monitored (for a particular protein/carbohydrate combination) by the degree of colour change that occurs as discussed above. An alternative measure is to assay the unreacted sugar.

Compositions contemplated by the present disclosure may further comprise additional components, for example, antioxidants, anti-caking agents, flavouring agents, colouring agents, vitamins, minerals, amino acids, chelating agents and the like.

Suitable antioxidants are well known to those skilled in the art, and may be water soluble or oil soluble. Suitable water soluble antioxidants include, for example, sodium ascorbate, calcium ascorbate, potassium ascorbate, arcorbic acid, glutathione, lipoic acid and uric acid. In an embodiment the water soluble antioxidant may be present in the composition in a range of about 0-10% wt/wt of the total composition. Suitable oil soluble antioxidants include, for example, tocopherols, ascorbyl palmitate, tocotrienols, phenols, polyphenols and the like. In an embodiment the oil soluble antioxidant is present in the oil phase in a range of about 0-10% wt/wt of the total composition.

Anti-caking agents that are compatible with the compositions of the present disclosure will be well known amongst those skilled in the art and include calcium phosphates, such as tricalcium phosphate and carbonates, such as calcium and magnesium carbonate and silicon dioxide

The compositions may further comprise one or more low molecular weight emulsifiers. Suitable low molecular weight emulsifiers include, for example, mono- and di-glycerides, lecithin and sorbitan esters. Other suitable low molecular weight emulsifiers will be well known to those skilled in the art. The low molecular weight emulsifier may be present in an amount between about 0.1% and 3% of the total weight of the composition, or in an amount between about 0.1% and about 2%, or in an amount between about 0.1% and 0.5%, or in an amount between about 0.1% and 0.3%, of the total weight of the composition.

Compositions contemplated herein may be formulated for administration to subjects by any suitable route, typically oral administration. The composition may be in liquid or solid form, and may be consumed as such (for example in the form of a syrup or other suitable liquid, or as capsules or other suitable solid form). Alternatively, the compositions may be incorporated into food or beverage products.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The present invention will now be further described in greater detail by reference to the following specific examples, which should not be construed as in any way limiting the scope of the invention.

### EXAMPLES

### Example 1

### Encapsulation of Phospholipid-Containing Oils in the Presence of a Hydrocolloid

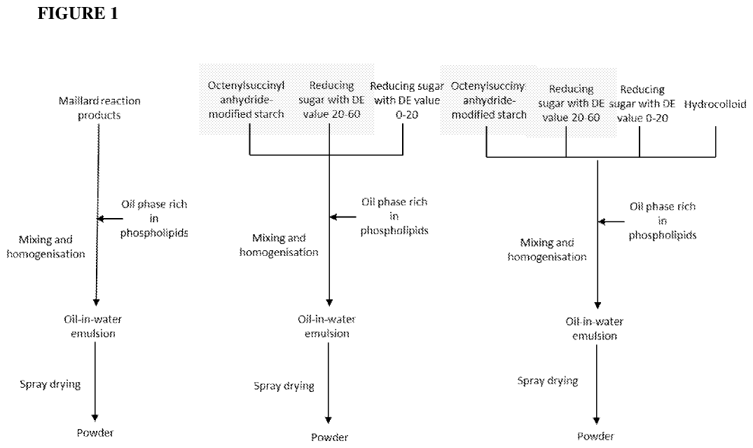

A phospholipid-rich hill oil (with a phospholipid content greater than 56%) was encapsulated using either a protein-based Maillard reaction products (MRP system) or an octenylsuccinyl anhydride-modified starch-based matrix system, with or without a hydrocolloid (xanthan gum) into an oil-in-water emulsion, followed by spray drying. The stability of the emulsion and surface free fat content of the spray dried powder was studied to evaluate the effectiveness of the encapsulation systems. The process flow is shown in FIG. 1.

With reference to FIG. 1, in the MRP system, aqueous MRPs were heated to 50-80° C. and mixed with the krill oil at 6,000-12,000 rpm for 5 min, followed by homogenisation at 350/100 bars by 1 pass to prepare a phospholipid-rich oil-in-water emulsion. The emulsion was further spray dried at an inlet temperature of 180° C. and outlet temperature of 80° C. to produce the final powder product. In the octenylsuccinyl anhydride-modified starch-based matrices without hydrocolloids, octenylsuccinyl anhydride-modified starch and sugars with reducing groups were hydrated under agitation (300-700 rpm for 30-60 min) in the temperature range of 50-80° C. to prepare the encapsulant slurry. The krill oil was mixed with this encapsulant slurry and homogenised at 6,000-12,000 rpm for 5 min, followed by homogenisation at 350/100 bars by 1 pass to prepare a phospholipid-rich oil-in-water emulsion. The obtained emulsion was then spray dried as described above for the MRP system. In the octenylsuccinyl anhydride-modified starch-based matrices with hydrocolloids, xanthan gum was added into the encapsulant slurry at the dosage of 0.1% to 0.5% w/w (with respect to water content). The hill oil was mixed with the encapsulant slurry at 6,000-12,000 rpm for 5 min, followed by homogenisation at 350/100 bars by 1 pass to prepare a phospholipid-rich oil-in-water emulsion. The emulsion was finally spray dried as described above for the MRP system. The compositions produced using the octenylsuccinyl anhydride-modified starch-based matrix system in the presence of xanthan gum are detailed in Table 1 below. From left to right, the formulations detailed in Table 1 contain 0.1%, 0.2%, 0.3%, 0.4% and 0.5% xanthan gum (w/w relative to water content).

TABLE 1
Microencapsulated krill oil powder formulations with xanthan
gum content of 0.1%-0.5% w/w (xanthan/water)

| Component | Weight (g) | | | | |
|---|---|---|---|---|---|
| Krill oil (with | 60.58 | 60.58 | 60.19 | 60.19 | 60.58 |
| phospholipid | | | | | |
| content >56%) | | | | | |
| C*Dry MD 010960 | 96.53 | 95.75 | 95.66 | 95.18 | 96.50 |
| (Maltodextrin 10) | | | | | |
| Purity gum 2000 | 11.30 | 11.21 | 11.2 | 11.14 | 11.30 |
| Dridex 30 | 23.22 | 23.03 | 23.01 | 22.9 | 23.22 |
| (maltodextrin 30) | | | | | |
| Tricalcium phosphate | 0.97 | 0.96 | 0.96 | 0.96 | 0.97 |
| Citric acid | 0.009 | 0.009 | 0.009 | 0.009 | 0.009 |
| Sodium ascorbate | 8.82 | 8.75 | 8.74 | 8.7 | 8.82 |
| Xanthan gum | 0.55 | 1.23 | 1.80 | 2.39 | 3.02 |
| Water | 604 | 601 | 599 | 597 | 604 |

Prior to spray drying, the physical stability of the prepared hill oil-in-water emulsions (see Table 1) was investigated because a stable spray dried can only be produced from an emulsion with good stability. As shown in Table 2 below, the MRPs-stabilised hill oil-in-water emulsion did not exhibit good stability. Specifically, “creaming” was observed within 48 hours after preparation due to the lipids which were not stabilised by MRPs, but no oil/water phase separation occurred. Using the octenylsuccinyl anhydride-modified starch-based matrix system in the absence of hydrocolloids, the oil-in-water emulsion remained stable at a lower solid content (<15%) compared to the MRP system. However with a solid content above 20%, the hill oil was not stable in the emulsion, likely due to the high viscosity imparted by the high phospholipid content in the oil; oil/water phase separation was observed within 48 hours. The viscosity of the emulsion increased with an increase of the xanthan gum content and this resulted in improved emulsion stability (Table 2). Thus, the addition of xanthan gum at 0.1%-0.5% w/w xanthan gum with respect to the water content resulted in superior physical stability of krill oil-in-water emulsions.

TABLE 2
Emulsion stability
Emulsion stability of krill oil-in-water emulsion with oil loading of 30%

| | | | Octenyl- | Octenyl- | Octenyl- |
|---|---|---|---|---|---|
| | | Octenyl- | succinyl | succinyl | succinyl |
| | | succinyl | anhydride- | anhydride- | anhydride- |
| | | anhydride- | modified | modified | modified |
| | | modified | starch- | starch- | starch- |
| | | starch- | based | based | based |
| Solid | MRPs | based | matrix + | matrix + | matrix + |
| content | system | matrix | 0.1% XAN | 0.3% XAN | 0.5% XAN |
| 12% | − | + | + | + | + |
| 15% | − | + | + | + | + |
| 20% | − | − | + | + | + |
| 25% | − | − | + | + | + |

XAN = xanthan gum
“−” indicates that phase separation occurred with 48 hours of preparation
“+” indicates a stable emulsion beyond 48 hours after preparation

Subsequently, the krill oil-in-water emulsions (30% oil loading, 25% solid content) stabilised by the MRP system or the octenylsuccinyl anhydride-modified starch-based matrix system in the presence of 0.5% xanthan gum w/w (xanthan gum/water) were spray dried to produce krill oil powder and the surface free fat content (SFF) was analysed to evaluate the effectiveness of the encapsulation system. Data for the octenylsuccinyl anhydride-modified starch-based matrix system in the absence of xanthan gum was not available at 25% solid content because of the poor stability of the prepared emulsion.

The surface free fat content of the hill oil microcapsule in the MRP system and in the octenylsuccinyl anhydride-modified starch-based matrix system in the presence of xanthan gum was analysed according to the method of Kim, E. H.-J. et al. (2005) Melting characteristics of fat present on the surface of industrial spray-dried dairy powders, Colloids and Surfaces B: Biointerfaces, 42:1-8, with minor modification. Briefly, 1 g of fresh trial powder was weighted on a filter paper (No. 541, Whatman, Maidstone, Kent, UK), and washed with 1×5 ml of petroleum ether. After also washing a funnel with petroleum ether, the solvent in the filtrate solution containing the extracted fat was evaporated until the extracted fat residue achieved a constant weight. The ratio of extracted fat value and weight of trial powder (i.e., 1 g) was recorded as surface free fat (%, g/g). As shown in Tables 3 and 4, the spray dried hill oil powder in the octenylsuccinyl anhydride-modified starch-based matrix system with from 0.1% to 0.5% w/w xanthan gum (with respect to water content) exhibited a significantly decreased surface free fat content, compared to the MRP system.

TABLE 3
Surface free fat content of krill oil encapsulation powder
Spray dried krill oil microcapsule with 30% oil loading

| | | Octenylsuccinyl anhydride- |
|---|---|---|
| | | modified starch-based |
| | MRPs | matrix + 0.5% XAN |
| Surface free fat content | >10% (w/w) | 1.4% (w/w) |

TABLE 4
Surface free fat content of krill oil encapsulation powder
at 0.1% to 0.5% w/w xanthan gum with respect to water content

| Xanthan gum (%) | Surface free fat (%) |
|---|---|
| 0.1 | 1.5 |
| 0.2 | 1.4 |
| 0.3 | 1.3 |
| 0.4 | 1.5 |
| 0.5 | 1.4 |

### Example 2

### Shelf Life of Phospholipid-Containing Oils in the Presence of a Hydrocolloid

Spray dried powder comprising 0.3% w/w xanthan gum with respect to water content, prepared as described in Example 1 (see Table 1), was stored at 40° C. in the presence of modified atmosphere (N₂) in sealed bags over 24 weeks. After the extraction of the stabilised oil from the powder, a series of oxidative parameters, including peroxide value (PoV), p-anisidine value (p-AV) and DHA and EPA content, were monitored every six weeks throughout the storage period. Peroxide value (PoV) and p-anisidine value (p-AV) are accepted indicators for the production of primary and secondary oxidation products.

In order to analyse the oxidative stability of the stabilised oil phase in the encapsulant, the entrapped oil was extracted and its peroxide value (PoV) and p-Anisidine value (p-AV) were determined. Generally, PoV is the measure of primary oxidation in a lipid and it reflects the oxidation and indicates possible future secondary oxidation. However, it is also possible to have a PoV close to zero for severely oxidised lipids because the hydro-peroxides measured by the PoV are easily broken down or are consumed to form secondary oxidation products. Hence, p-AV is usually used as an indicator for secondary oxidation products, mainly unsaturated aldehyde compounds, to reflect the secondary oxidation that has taken place. Meanwhile, the polyunsaturated fatty acid such as DHA and EPA active contents of the stabilised oil phase are desired to remain unchanged during the shelf life of product.

In the Example 2, the PoV of the extracted oil was analysed based on AOAC Official Method 965.33. The extracted oil was mixed with an acetic acid-chloroform solution and titrated with sodium thiosulphate solution after addition of potassium iodide. In the test, the starch indicator was used and the titration stopped once the colour was changed. The p-AV of the extracted oil was determined based on AOCS Official Method Cd 18-90. Briefly, the extracted oil was diluted by isooctane, followed by reaction with p-anisidine in the acetic acid solution. The formed conjugates were quantified by its absorbance at 350 nm. Global Organization for EPA and DHA Omega-3 (GOED) recommdeds that the PoV and p-AV of the edible oils should not exceed 5 mgq/kg and 20, respectively. The DHA and EPA active content in the extracted oil was quantified using gas chromatography-flame ionization detector (GC-FID) technique, according to AOAC Official Method 996.06. Briefly, the extracted oil was esterified and the formed methyl esters were extracted and pre-filtered to remove the moisture content. The fatty acid methyl esters were separated and quantified using gas chromatography and quantified by GC-FID, equipped with a specified column.

Results are shown in Table 5. During 24 weeks storage, the PoV and p-AV remained unchanged and both were below the maximum acceptable limit recommended by the Global Organization for EPA and DHA Omega-3 (GOED) for general food products. Furthermore, the DHA and EPA active content varied little.

TABLE 5
Oxidative parameters of microencapsulated krill oil at 40° C. in
sealed package during 24 weeks storage
Oxidative parameters of microencapsulated krill oil

| Oxidative parameter | 0 week | 6 week | 12 week | 18 week | 24 week |
|---|---|---|---|---|---|
| Peroxide value | 0.2 | 0.5 | 0.2 | 0.2 | 0.7 |
| (PoV, mEq/Kg) | | | | | |
| Max acceptable peroxide | | | | <5 | |
| value (PoV)¹ | | | | | |
| p-anisidine value | 2 | 3.3 | 5.2 | 6.1 | 5 |
| (p-AV) | | | | | |
| Max acceptable | | | | <20 | |
| p-anisidine value | | | | | |
| (p-AV)¹ | | | | | |
| DHA (mg/g) | 27.8 | 27.1 | 25.4 | 24.3 | 27.5 |
| Target DHA (mg/g) | | | | >19.8 | |
| EPA (mg/g) | 47.2 | 46.6 | 43.1 | 40.7 | 45.7 |
| Target EPA (mg/g) | | | | >36.7 | |

¹Maximum acceptable PoV and p-AV were recommended by Global Organization for EPA and DHA Omega-3 (GOED).

1. An encapsulated oil or lipid composition comprising one or more long chain polyunsaturated fatty acids (LCPUFAs) and at least one hydrocolloid, wherein the composition has a surface free fat content of less than about 5%, and wherein the hydrocolloid is present in a concentration of between about 0.05% to about 1% w/w with respect to the amount of water in the composition.

2. (canceled)

3. (canceled)

4. The composition according to claim 1, wherein the composition is in the form of an emulsion or a powder.

5. The composition according to claim 4, wherein the emulsion is an oil-in-water emulsion.

6. (canceled)

7. (canceled)

8. The composition according to claim 1, wherein the oil or lipid composition comprises at least 5-55% phospholipids.

9. The composition according to claim 1, wherein the oil comprises hill oil or a fish oil.

10.-12. (canceled)

13. The composition according to claim 1, wherein the hydrocolloid is xanthan gum.

14. (canceled)

15. The composition according to 1, wherein the one or more LCPUFAs or the oil or lipid composition comprising the one or more LCPUFAs is encapsulated with an octenylsuccinic anhydride-modified starch and two or more sources of reducing sugars.

16. The composition according to claim 15, wherein one of said sources of reducing sugars has a dextrose equivalent (DE) value of between 20 and 60, and a second of said sources of reducing sugars has a DE value of between about 0 and 20.

17. A method for increasing the efficiency of encapsulation of a composition comprising one or more LCPUFAs, the method comprising incorporating at least one hydrocolloid into said composition, wherein the hydrocolloid is present in a concentration of between about 0.05% to about 1% w/w with respect to the amount of water in the composition.

18. The method according to claim 17, wherein the composition is an oil or lipid composition comprising the one or more LCPUFAs.

19. The method according to claim 17, wherein the efficiency of encapsulation is determined and/or quantified by the surface free fat content of the encapsulated composition, compared to the surface free fat content in the absence of the at least one hydrocolloid.

20. The method according to claim 19, wherein the surface free fat content of the composition, in the presence of the at least one hydrocolloid is less than about 5% or less than about 2%.

21. The method according to claim 17, wherein the encapsulant comprises an octenylsuccinic anhydride-modified starch and two or more sources of reducing sugars.

22.-24. (canceled)

25. The method according to claim 17, wherein the at least one hydrocolloid comprises xanthan gum.

26. (canceled)

27. A method for stabilising an emulsion comprising one or more LCPUFAs, the method comprising incorporating at least one hydrocolloid into said emulsion wherein the hydrocolloid is present in a concentration of between about 0.05% to about 1% w/w with respect to the amount of water in the emulsion.

28. The method according to claim 27, wherein the emulsion comprises an oil or lipid composition comprising the one or more LCPUFAs.

29. The method according to claim 27, wherein the wherein the surface free fat content of the emulsion in the presence of the at least one hydrocolloid is less than about 5% or less than about 2%.

30. The method according to claim 27, wherein the one or more LCPUFAs or the oil or lipid composition comprising the one or more LCPUFAs is encapsulated with an octenylsuccinic anhydride-modified starch and two or more sources of reducing sugars.

31.-32. (canceled)

33. The method according to claim 27, wherein the at least one hydrocolloid comprises xanthan gum.

34.-35. (canceled)

36. A stable emulsion comprising one or more LCPUFAs and at least one hydrocolloid, wherein the hydrocolloid is present in a concentration of between about 0.05% to about 1% w/w with respect to the amount of water in the emulsion.

37. The stable emulsion according to claim 36, wherein the emulsion is an oil-in-water emulsion.

38.-39. (canceled)

40. The stable emulsion according to claim 36, wherein the at least one hydrocolloid comprises xanthan gum.

41.-43. (canceled)
